(19) United States
(12) Patent Application Publication
(10) Pub. No.: US 2019/0029762 A1
(43) Pub. Date: Jan. 31, 2019

(54) MEDICAL MANIPULATOR SYSTEM AND MANIPULATOR CURVED-SHAPE ESTIMATION METHOD

(71) Applicant: OLYMPUS CORPORATION, Tokyo (JP)

(72) Inventors: Masatoshi IIDA, Tokyo (JP); Naoya HATAKEYAMA, Tokyo (JP)

(73) Assignee: OLYMPUS CORPORATION, Tokyo (JP)

(21) Appl. No.: 16/148,256

(22) Filed: Oct. 1, 2018

Related U.S. Application Data
(63) Continuation of application No. PCT/JP2016/061196, filed on Apr. 6, 2016.

(51) Int. Cl. A61B 34/20; A61B 17/29; A61B 34/00

(52) U.S. Cl. CPC A61B 34/20; A61B 2017/0034; A61B 34/71; A61B 17/29

(57) ABSTRACT

A medical manipulator system includes: an elongated manipulator that has an insertion portion; a shape sensor that is provided so as to be movable along the longitudinal direction of a path in which the manipulator is to be disposed and that detects shape information of the path at respective positions; a shape estimating unit that is configured to estimate a curved shape of the manipulator on the basis of the shape information detected by the shape sensor and longitudinal-direction position information of the shape sensor in the path; and a controller that is configured to control the manipulator on a basis of the curved shape of the manipulator estimated by the shape estimating unit.

### CROSS-REFERENCE TO RELATED APPLICATIONS

This is a continuation of International Application PCT/JP2016/061196, with an international filing date of Apr. 6, 2016, which is hereby incorporated by reference herein in its entirety.

### TECHNICAL FIELD

The present invention relates to a medical manipulator system and a manipulator curved-shape estimation method.

### BACKGROUND ART

There is a known medical manipulator system in which a medical manipulator is inserted into the body of a patient through a forceps channel or the like of an endoscope inserted into a body cavity of the patient, to preform treatment on an affected area (for example, see PTL 1).

Although the medical manipulator is curved according to the shape of a tortuous body cavity of the patient, because the curved shape affects the operability of the medical manipulator, it is necessary to accurately detect the curved shape.

In the medical manipulator system of PTL 1, an optical fiber sensor that is constituted of a fiber bundle formed of a plurality of optical fibers is disposed along the longitudinal direction of the medical manipulator, and the bend radius of the medical manipulator is detected at a plurality of places along the longitudinal direction of the medical manipulator.

### CITATION LIST

### Patent Literature

{PTL 1} U.S. Pat. No. 7,720,322

### SUMMARY OF INVENTION

One aspect of the present invention is directed to a medical manipulator system including: an elongated manipulator that has an insertion portion; a shape sensor that is provided so as to be movable along the longitudinal direction of a path in which the manipulator is to be disposed and that detects shape information of the path at respective positions; a shape estimating unit that estimates a curved shape of the manipulator on the basis of the shape information detected by the shape sensor and longitudinal-direction position information of the shape sensor in the path; and a controller that is configured to control the manipulator on a basis of the curved shape of the manipulator estimated by the shape estimating unit.

Another aspect of the present invention is directed to a manipulator curved-shape estimation method including: moving a shape sensor along the longitudinal direction of a path in which an elongated manipulator having an insertion portion is to be disposed; and estimating a curved shape of the manipulator on the basis of respective longitudinal-direction positions and shape information of the path detected by the shape sensor at the longitudinal-direction positions.

### BRIEF DESCRIPTION OF DRAWINGS

FIG. 1 is a view of the overall configuration of a medical manipulator system according to an embodiment of the present invention.

FIG. 2 is a view for explaining a manipulator curved-shape estimation method using the medical manipulator system shown in FIG. 1 and showing the states in which a sensor unit of a shape sensor is disposed at (a) a first position, (b) a second position, (c) a third position, and (d) a fourth position.

FIG. 3 is a view showing a first modification in which driving and position detection of the shape sensor of the medical manipulator system shown in FIG. 1 are performed.

FIG. 4 is a view showing a second modification in which driving and position detection of the shape sensor of the medical manipulator system shown in FIG. 1 are performed.

FIG. 5 is a view of a third modification of the shape sensor of the medical manipulator system shown in FIG. 2, in which a marker is provided on a flexible portion.

FIG. 6 is a perspective view showing a modification of a movement method for the shape sensor of the medical manipulator system shown in FIG. 1.

FIG. 7 is a perspective view showing a modification of the movement method for the shape sensor of the medical manipulator system shown in FIG. 1.

FIG. 8 is a perspective view of a modification of the shape sensor of the medical manipulator system shown in FIG. 1, showing example movement trajectories of the shape sensor when two sensor units are provided.

### DESCRIPTION OF EMBODIMENT

A medical manipulator system 1 and a curved-shape estimation method for a manipulator 2 according to an embodiment of the present invention will be described below with reference to the drawings.

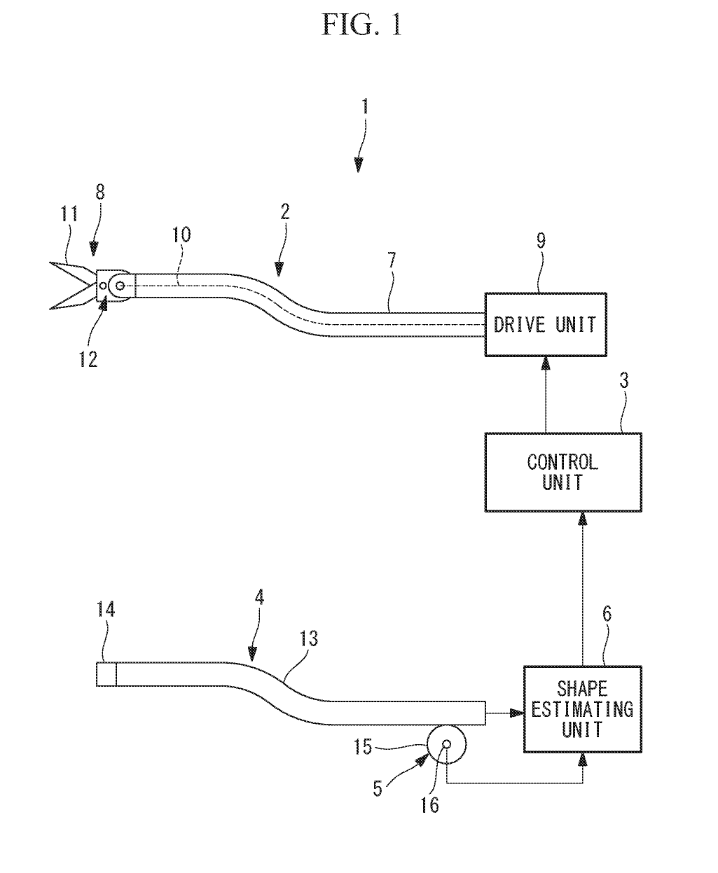

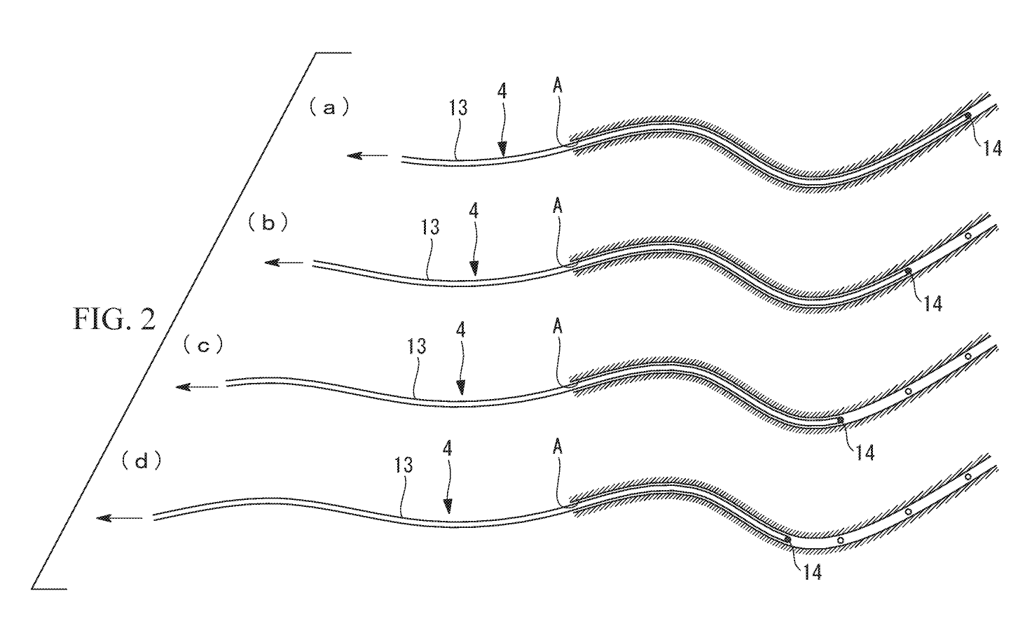

As shown in FIGS. 1 and 2, the medical manipulator system 1 of this embodiment is provided with: the manipulator 2; a control unit 3 that controls the manipulator 2; a shape sensor 4 that detects the radius of curvature (shape information) of a path A; an insertion-amount detecting unit 5 that detects an insertion amount of the shape sensor 4 into the path A; and a shape estimating unit 6 that estimates the curved shape of the manipulator 2 on the basis of the radius of curvature detected by the shape sensor 4 and the insertion amount detected by the insertion-amount detecting unit 5.

The manipulator 2 is provided with: an elongated flexible insertion portion 7; a movable portion 8 that is disposed at a distal end of the insertion portion 7; a drive unit 9 that is provided at a base end of the insertion portion 7; and a power transferring member, such as a wire 10, that transfers power from the drive unit 9 to the movable portion 8.

The movable portion 8 is provided with: a treatment part 11, such as grasping forceps, that is disposed at the distal end thereof; and a joint 12 that changes the orientation of the treatment part 11.

The drive unit 9 is provided with a motor (not shown) for generating power and generates a tension in the wire 10 through rotation of the motor, thus causing the treatment part 11 and the joint 12 to move due to the tension.

The shape sensor 4 is a single optical fiber sensor, for example, and detects, at a sensor unit (shape sensor) 14 that is disposed at the distal end of an elongated flexible portion (elongated member) 13, a signal corresponding to the radius of curvature in the direction along one plane including the longitudinal axis of the flexible portion 13.

The insertion-amount detecting unit 5 is provided with: a roller (drive part) 15 that is in contact with the outer surface of the flexible portion 13 of the shape sensor 4 and that is rotated through movement of the flexible portion 13 in the longitudinal direction; an encoder (position sensor) 16 that detects a rotation amount of the roller 15; and a position-information calculating unit (not shown) that detects a movement amount (longitudinal-direction position information) of the shape sensor 4 in the longitudinal direction on the basis of the rotation amount detected by the encoder 16.

The shape estimating unit 6 estimates, while the shape sensor 4 is moved along the longitudinal direction of the path A, where the manipulator 2 is to be disposed, the curved shape of the path A on the basis of the insertion amount detected by the insertion-amount detecting unit 5 and the signal corresponding to the radius of curvature detected by the shape sensor 4.

Here, the path A means a channel that is provided in an endoscope or an overtube into which the manipulator 2 is inserted, a channel that is provided in the manipulator 2 itself, or an inner hole of a tube that is disposed side by side with the outer surface of the manipulator 2. In particular, when the manipulator 2 is inserted into a tortuous body cavity of a patient to perform treatment on an affected area in the body, the path A means an arbitrary path indicating the curved shape of the manipulator 2 being inserted into the body cavity.

In order to estimate the curved shape of the path A using the shape estimating unit 6, in a state where the shape sensor 4 is inserted into the path A, as shown in FIG. 2(a), the shape sensor 4 is moved in the direction in which the shape sensor 4 is pulled out from the path A along the longitudinal direction of the path A, as shown in FIGS. 2(b) to 2(d), a signal corresponding to the radius of curvature is detected at each position in the longitudinal direction where the sensor unit 14 is located, and the detected signal corresponding to the radius of curvature is associated with the longitudinal-direction position information. Accordingly, the curved shape of the path A can be estimated so as to be traced by the sensor unit 14.

The control unit 3 adjusts a control parameter of the drive unit 9 on the basis of the curved shape of the manipulator 2 estimated by the shape estimating unit 6. Specifically, the drive unit 9 is controlled such that a larger tension is generated in the wire 10 as the magnitude of the total curve angle of the manipulator 2 is increased.

The operation of the thus-configured medical manipulator system 1 of this embodiment will be described below.

In order to perform treatment in the body of a patient by using the medical manipulator system 1 of this embodiment, for example, the shape sensor 4 is inserted, starting from the sensor unit 14, into a forceps channel in an endoscope that has been inserted up to the vicinity of an affected area along the tortuous body cavity of the patient. For example, the sensor unit 14 is inserted up to a first position at which the sensor unit 14 is included in the field of view of an objective lens of the endoscope.

From this state, the estimation method for the curved shape of the manipulator 2 according to the embodiment of the present invention is performed. The curved-shape estimation method of this embodiment includes a step of measuring the radius of curvature by means of the shape sensor 4 (shape measurement step), a step of moving the shape sensor 4 in the longitudinal direction (movement step), these steps being repeated, and a step of estimating the curved shape of the path A on the basis of data in which a plurality of obtained longitudinal-direction positions and radii of curvature are associated (shape estimation step).

Specifically, first, at the first position, detection light is emitted from the base end of the optical fiber sensor, which constitutes the shape sensor 4, and light returning to the base end is analyzed, thereby obtaining the radius of curvature of the forceps channel at the sensor unit 14 (shape measurement step). Next, the shape sensor 4 is moved along the longitudinal direction in the direction in which the shape sensor 4 is pulled out from the path A by a predetermined amount (movement step), and the radius of curvature is obtained at a second position (shape measurement step). On the assumption that the radius of curvature from the first position to the second position is constant at the magnitude of the radius of curvature at the first position, the curve angle θ can be calculated by using Expression (1).

θ=S/R (1)

where S indicates the length of an arc between the first position and the second position, and R indicates the radius of curvature.

The length S of the arc is equal to the movement amount (insertion amount) in the longitudinal direction between the first position and the second position, which is detected by the insertion-amount detecting unit 5, and the radius of curvature R can be detected by the shape sensor 4.

Therefore, the curve angle θ between the first position and the second position can be calculated by using the obtained S and R (shape estimation step).

Next, the shape sensor 4 is moved along the longitudinal direction in the direction in which the shape sensor 4 is pulled out from the path A by the predetermined amount (movement step), and the radius of curvature is obtained at a third position (shape measurement step). On the assumption that the radius of curvature from the second position to the third position is constant at the magnitude of the radius of curvature at the second position, the curve angle θ from the second position to the third position can be calculated by using Expression (1).

These steps are repeated, and the curve angles θ in a plurality of sections are obtained and are added up, thus making it possible to obtain the total curve angle (shape estimation step).

In this way, according to the medical manipulator system 1 and the curved-shape estimation method for the manipulator 2 of this embodiment, because the curved shape of the path A is estimated by detecting shape information at the respective positions while moving a small number of the shape sensors 4 in the longitudinal direction of the path A, there is an advantage in that a larger number of shape sensors 4 is not required, and the curved shape of the manipulator 2 along the longitudinal direction can be measured by using the minimum number of shape sensors 4.

Then, instead of the shape sensor 4, the manipulator 2 is inserted into the path A whose curved shape has been estimated by moving the shape sensor 4 in this way, thereby curving the manipulator 2 according to the curved shape of the path A. Accordingly, there is an advantage in that the control unit 3 controls the manipulator 2 on the basis of the estimated curved shape of the path A, thereby making it possible to improve the operability of the manipulator 2.

Note that, in this embodiment, although measurement is started from a state in which the shape sensor 4 is inserted up to the distal end of the endoscope, and the curved shape is calculated while the shape sensor 4 is pulled out along the longitudinal direction of the forceps channel, instead of this, it is also possible to calculate the curved shape while the shape sensor 4 is inserted along the longitudinal direction of the forceps channel. In this case, it is also possible to dispose, at an inlet of the forceps channel, an insertion sensor (not shown) that detects the start of insertion and to detect an insertion start position. Accordingly, the shape of the forceps channel can be more accurately estimated.

Furthermore, although the movement amount of the flexible portion 13 of the shape sensor 4 in the longitudinal direction is converted into the rotation amount by the roller 15, which rolls on the outer surface of the flexible portion 13, and the encoder 16 detects the rotation amount, thus detecting the movement amount of the shape sensor 4 in the longitudinal direction, instead of this, it is also possible to dispose a plurality of magnets (not shown) at intervals on the shape sensor 4 in the longitudinal direction thereof and to detect and count the magnetic force by means of a hall element (not shown) disposed in the vicinity of the inlet of the forceps channel, thereby detecting the movement amount of the shape sensor 4 in the longitudinal direction.

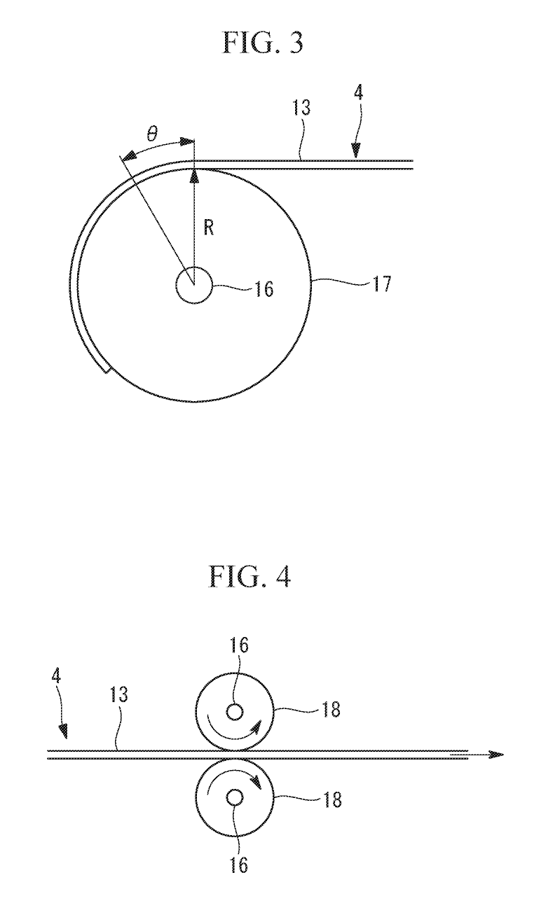

Furthermore, instead of the roller 15, which rolls on the outer surface of the flexible portion 13, it is also possible to provide a pulley (drive part) 17 that rolls up the flexible portion 13 of the shape sensor 4, as shown in FIG. 3, and to detect the movement amount of the flexible portion 13 in the longitudinal direction on the basis of the rotation amount of the pulley 17 detected by the encoder 16. By doing so, movement of the shape sensor 4 can also be automatically performed by rotation of the pulley 17.

Furthermore, in this embodiment, movement of the shape sensor 4 in the longitudinal direction can be manually performed or the shape sensor 4 can be automatically rolled up by the pulley 17 shown in FIG. 3; however, as shown in FIG. 4, the shape sensor 4 may also be automatically inserted into or pulled out from the forceps channel through rotation of rollers (drive part) 18 that sandwich the flexible portion 13 of the shape sensor 4 in a radial direction.

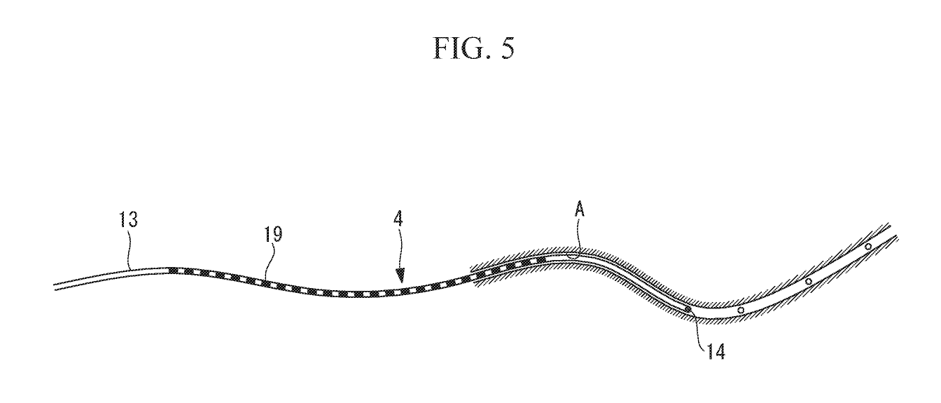

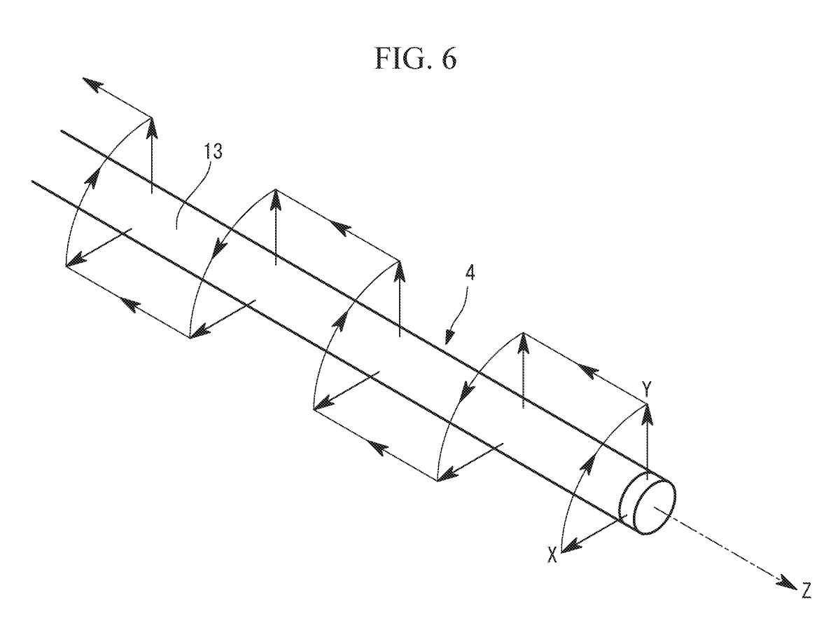

Furthermore, the movement amount of the shape sensor 4 in the longitudinal direction may be detected by the encoder 16, or, as shown in FIG. 5, it is also possible to provide a marker 19 serving as a scale on the flexible portion 13 along the longitudinal direction, and an operator may read the marker 19 and input the read marker.

Furthermore, in this embodiment, the shape sensor 4 is formed of a single optical fiber sensor, and the radius of curvature in the direction along one plane including the longitudinal axis of the flexible portion 13 at the position of the sensor unit 14 is detected.

However, because the path A, into which the manipulator 2 is inserted, is not curved in one plane but is three-dimensionally curved, it is not enough to detect the radius of curvature in the direction of one plane.

Thus, for example, as shown in FIG. 6, at the respective positions of the path A in the longitudinal direction, at each of which the sensor unit 14 is disposed, the shape sensor 4 may be rotated about the longitudinal axis of the path A by 90 degrees by using a rotary drive part (not shown) that rotates the shape sensor 4 about the longitudinal axis of the path A, thus obtaining the radii of curvature in two directions at 90 degree intervals in the circumferential direction. FIG. 6 shows an operating trajectory of the shape sensor 4 obtained when the shape sensor 4 detects the radii of curvature in the X-direction and the Y-direction at the first position, is then moved to the second position, and detects the radii of curvature in the X-direction and the Y-direction, and thereafter, the same operations are sequentially repeated.

Accordingly, because the radii of curvature in two directions that intersect with each other are obtained at the respective positions in the longitudinal direction, it is possible to obtain, through calculation, a 3D radius of curvature having these radii of curvature as components.

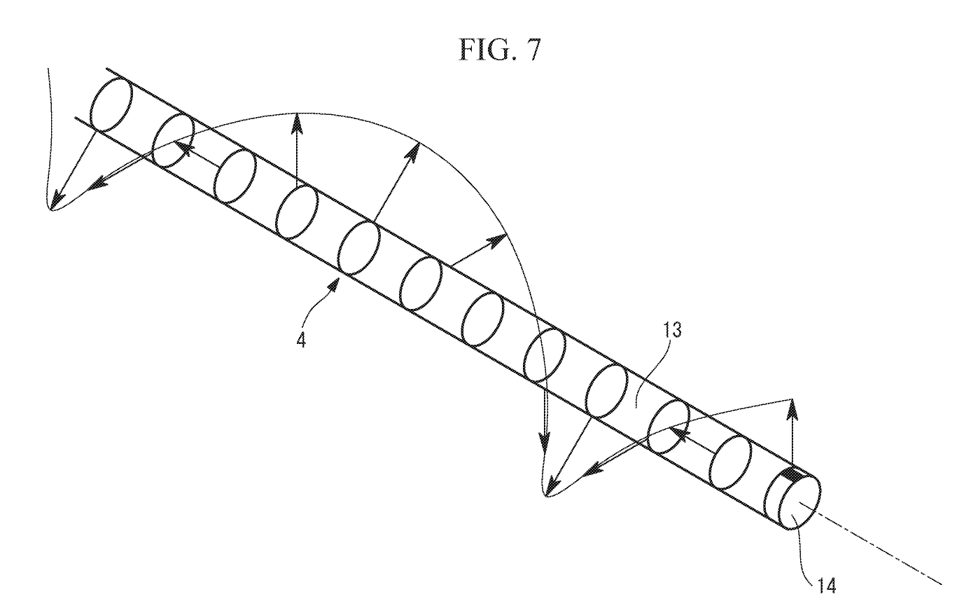

Instead of this, as shown in FIG. 7, it is also possible to move the sensor unit 14 in a spiral manner by rotating the sensor unit 14 about the longitudinal axis by using the rotary drive part while moving the shape sensor 4 in the longitudinal direction and to measure the radii of curvature in different radial directions at respective longitudinal-direction positions. Although the accuracy is less than in the case shown in FIG. 6, there is an advantage in that measurement can be performed in a short time.

Furthermore, although a description has been given of a case in which the sensor unit 14, which can detect the radius of curvature in one direction, is provided as the shape sensor 4, instead of this, for example, it is also possible to provide a sensor unit 14 that allows simultaneous measurement in two directions that are perpendicular to the longitudinal axis of the flexible portion 13 and that intersect with each other (preferably, that are perpendicular to each other). In this case, the medical manipulator system 1 is provided with a curvature-radius calculating unit (not shown) that calculates a 3D radius of curvature of the path A on the basis of the radii of curvature in the two directions detected by the shape sensor 4.

By doing so, it is possible to simultaneously detect the radius-of-curvature components in two directions at respective positions at each of which the shape sensor 4 is moved in the longitudinal direction of the path A and to obtain a 3D radius of curvature through calculation of the curvature-radius calculating unit.

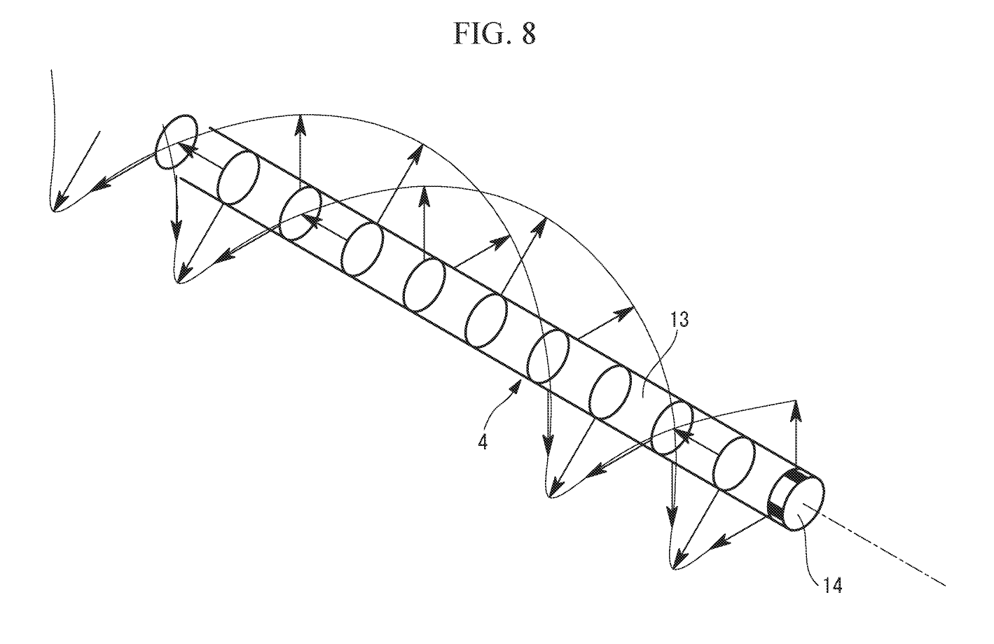

In particular, when the shape sensor 4 is moved along the longitudinal direction of the path A, the shape sensor 4 is moved while being rolled about the longitudinal axis thereof in many cases; however, if the radius-of-curvature components in two directions are simultaneously detected, as shown in FIG. 8, even when the detection directions of the sensor unit 14 change in the circumferential direction, the magnitude of a 3D radius of curvature can be accurately calculated. Accordingly, there is an advantage in that the curved shape of the manipulator 2 disposed in the path A can be accurately estimated.

Furthermore, in this embodiment, although the control unit 3 adjusts the control parameter according to the total curve angle of the path A estimated by the shape estimating unit 6, instead of this, it is also possible to calculate a friction force occurring in the wire 10 on the basis of the total curve angle and to control the drive unit 9 so as to produce a tension opposing the friction force. Furthermore, it is also possible to control the drive unit 9 by directly calculating a control compensation amount that can produce a tension opposing the friction force.

Furthermore, in this embodiment, although a description has been given of a case in which the shape sensor 4 is an optical fiber sensor that detects the radius of curvature of the path A, the present invention is not limited thereto, and the shape sensor 4 may be a sensor that detects a 3D position coordinate where the sensor unit 14 is disposed.

As a result, the above-described embodiment leads to the following aspects.

One aspect of the present invention is directed to a medical manipulator system including: an elongated manipulator that has an insertion portion; a shape sensor that is provided so as to be movable along the longitudinal direction of a path in which the manipulator is to be disposed and that detects shape information of the path at respective positions; a shape estimating unit that estimates a curved shape of the manipulator on the basis of the shape information detected by the shape sensor and longitudinal-direction position information of the shape sensor in the path; and a controller that is configured to control the manipulator on a basis of the curved shape of the manipulator estimated by the shape estimating unit.

According to this aspect, the shape sensor is disposed in a path in which the elongated manipulator, which has the insertion portion, is to be disposed, and the shape of the path is detected by the shape sensor while the shape sensor is moved in the longitudinal direction of the path, thereby making it possible to estimate the curved shape of the manipulator by means of the shape estimating unit on the basis of the detected shape of the path and longitudinal-direction position information thereof. Specifically, a small number of shape sensors are moved in the longitudinal direction, and the shape is detected at the respective positions in the longitudinal direction, thereby making it possible to measure the shape of the path, i.e., the curved shape of the manipulator along the longitudinal direction when the manipulator is disposed in the path, by using the minimum number of sensors, without using a shape sensor, such as a fiber bundle of a number of optical fibers.

In the above-described aspect, the shape sensor may detect the radius of curvature of the path.

By doing so, the shape sensor detects the radius of curvature of the path at the respective positions in the longitudinal direction, thereby making it possible to easily measure the curved shape of the path, i.e., the curved shape of the manipulator when the manipulator is disposed in the path, on the basis of the radii of curvature of the path at the plurality of positions.

Furthermore, the above-described aspect may further include a rotary drive part that rotates the shape sensor about the longitudinal axis of the path.

By doing so, even when the shape sensor is a sensor that can detect a radius of curvature only in a particular direction about the longitudinal axis, the rotary drive part rotates the shape sensor about the longitudinal axis, thereby making it possible to detect the radii of curvature in a plurality of directions about the longitudinal axis. Therefore, it is possible to estimate a 3D curved shape of the path and to measure the curved shape of the manipulator disposed in the path.

Furthermore, in the above-described aspect, the shape sensor may detect a 3D position in the path.

By doing so, a 3D curved shape of the path can be easily estimated on the basis of 3D position information detected by the shape sensor at a plurality of points of the path along the longitudinal direction.

Furthermore, the above-described aspect may further include a drive part that moves the shape sensor in the longitudinal direction of the path; and a position-information calculating unit that calculates longitudinal-direction position information of the shape sensor on the basis of a driving amount of the drive part.

By doing so, a 3D shape of the path can be easily estimated by detecting a plurality of combinations of a driving amount of the shape sensor along the longitudinal direction of the path driven by the drive part and shape information of the path detected by the shape sensor.

Furthermore, the above-described aspect may further include a position sensor that detects a longitudinal-direction position of the shape sensor.

By doing so, a 3D shape of the path can be easily estimated on the basis of a plurality of longitudinal-direction positions of the shape sensor detected by the position sensor and shape information of the path detected by the shape sensor at the respective longitudinal-direction positions.

Furthermore, in the above-described aspect, the shape sensor may be disposed at the distal end of a flexible elongated member; and the elongated member may be provided with a marker with which an insertion amount into the path can be measured.

By doing so, a 3D shape of the path can be easily estimated on the basis of insertion amounts read from the marker on the elongated member, to which the shape sensor is attached, and the shape information detected by the shape sensor at the positions corresponding to the respective insertion amounts.

Furthermore, the above-described aspect may further include an insertion sensor that detects the start of insertion of the shape sensor is provided at an inlet of the path.

By doing so, it is possible to accurately detect the position of the shape sensor on the basis of an insertion amount obtained after the start of insertion of the shape sensor is detected by the insertion sensor and to more accurately estimate the curved shape of the path.

Furthermore, the above-described aspect may further include the shape sensor detects the radii of curvature of the path in two directions that are perpendicular to the longitudinal axis of the path and that intersect with each other; and the medical manipulator system further comprises a curvature-radius calculating unit that calculates the radius of curvature of the path on the basis of the radii of curvature in the two directions detected by the shape sensor.

By doing so, it is possible to detect the radii of curvature of the path in two directions that are perpendicular to the longitudinal direction of the path and that intersect with each other and to calculate a 3D radius of curvature of the path that includes the detected radii of curvature in the two directions as components, by means of the curvature-radius calculating unit.

Another aspect of the present invention is directed to a manipulator curved-shape estimation method including: moving a shape sensor along the longitudinal direction of a path in which an elongated manipulator having an insertion portion is to be disposed; and estimating a curved shape of the manipulator on the basis of respective longitudinal-direction positions and shape information of the path detected by the shape sensor at the longitudinal-direction positions.

According to the present invention, an advantageous effect is afforded in that the curved shape of a medical manipulator along the longitudinal direction thereof can be measured by using the minimum number of sensors.

### REFERENCE SIGNS LIST

1 medical manipulator system
2 manipulator
4 shape sensor
6 shape estimating unit
7 insertion portion
13 flexible portion (elongated member)
14 sensor unit (shape sensor)
15 roller (drive part)
16 encoder (position-information calculating unit, position sensor)
17 pulley (drive part)
18 roller (drive part)
19 marker
A path

1. A medical manipulator system comprising:
an elongated manipulator that has an insertion portion;
a shape sensor that is provided so as to be movable along the longitudinal direction of a path in which the manipulator is to be disposed and that detects shape information of the path at respective positions;
a shape estimating unit that is configured to estimate a curved shape of the manipulator on the basis of the shape information detected by the shape sensor and longitudinal-direction position information of the shape sensor in the path; and
a controller that is configured to control the manipulator on a basis of the curved shape of the manipulator estimated by the shape estimating unit.

2. The medical manipulator system according to claim 1, wherein the shape sensor detects the radius of curvature of the path.

3. The medical manipulator system according to claim 2, further comprising a rotary drive part that rotates the shape sensor about the longitudinal axis of the path.

4. The medical manipulator system according to claim 1, wherein the shape sensor detects a 3D position in the path.

5. The medical manipulator system according to claim 1, further comprising:
a drive part that moves the shape sensor in the longitudinal direction of the path; and
a position-information calculating unit that calculates longitudinal-direction position information of the shape sensor on the basis of a driving amount of the drive part.

6. The medical manipulator system according to claim 1, further comprising a position sensor that detects a longitudinal-direction position of the shape sensor.

7. The medical manipulator system according to claim 1, wherein the shape sensor is disposed at the distal end of a flexible elongated member; and
the elongated member is provided with a marker with which an insertion amount into the path can be measured.

8. The medical manipulator system according to claim 1, wherein an insertion sensor that detects the start of insertion of the shape sensor is provided at an inlet of the path.

9. The medical manipulator system according to claim 1,
wherein the shape sensor detects the radii of curvature of the path in two directions that are perpendicular to the longitudinal axis of the path and that intersect with each other; and
the medical manipulator system further comprises a curvature-radius calculating unit that calculates the radius of curvature of the path on the basis of the radii of curvature in the two directions detected by the shape sensor.

10. A manipulator curved-shape estimation method comprising:
moving a shape sensor along the longitudinal direction of a path in which an elongated manipulator having an insertion portion is to be disposed; and
estimating a curved shape of the manipulator on the basis of respective longitudinal-direction positions and shape information of the path detected by the shape sensor at the longitudinal-direction positions.
